(19) United States
(12) Patent Application Publication
(10) Pub. No.: US 2025/0195778 A1
(43) Pub. Date: Jun. 19, 2025

(54) PRE-FILLED SYRINGE WITH INTEGRATED DISINFECTING FEATURE

(71) Applicant: Becton, Dickinson and Company, Franklin Lakes, NJ (US)

(72) Inventors: Roza Mahmoodian, New York, NY (US); Jeremy Ridley, Philadelphia, PA (US); David R. Schiff, Highland Park, NJ (US); Donald Fraser VanRoyen, Philadelphia, PA (US)

(73) Assignee: Becton, Dickinson and Company, Franklin Lakes, NJ (US)

(21) Appl. No.: 18/981,867

(22) Filed: Dec. 16, 2024

Related U.S. Application Data
(60) Provisional application No. 63/612,033, filed on Dec. 19, 2023.

(51) Int. Cl. A61M 5/31; A61M 5/315

(52) U.S. Cl. CPC A61M 5/3134; A61M 5/31505; A61M 2005/3117

(57) ABSTRACT

A flush syringe and integrated swab assembly comprises a chamber and a tip having a passageway. The assembly includes a plunger rod and an integral swab including an integral collar extending from the distal wall of the barrel, surrounding the distal tip, and having a collar distal end and a collar proximal end, the integral collar including at least one sidewall having an inside surface including an inner thread thereon defining a Luer lock area and configured to connect to the hub of a vascular access device connector, the at least one sidewall further having an outside surface including an outer thread thereon configured for connection to the removable cap that when connected to the integral collar covers the integral collar to protect the distal tip and the collar from contamination, and a porous hollow mass connected to the inner thread of the integral collar.

### TECHNICAL FIELD

The present disclosure claims priority to U.S. Provisional Application No. 63/612,033, filed Dec. 19, 2023, the entire disclosure of which is hereby incorporated by reference herein.

### TECHNICAL FIELD

The present disclosure pertains to syringes, and particularly to syringes comprising an integrated disinfecting feature.

### BACKGROUND

Vascular access devices (VAD's) are commonly used therapeutic devices and include intravenous (IV) catheters. There are two general classifications of VAD's, peripheral catheters and central venous catheters. To ensure VAD's are used and maintained correctly, standards of practice have been developed, which include a cleaning procedure, commonly referred to as flushing a catheter.

VAD standards of practice usually recommend that flush procedures be performed after catheter placement, before fluid infusion, and before and after drug administration, blood sampling, transfusions and parenteral nutrition. The goal of these flush procedures is to confirm catheter patency, avoid drug incompatibilities, ensure complete drug dose administration, prevent thrombus formation and minimize the risk of blood stream infections. Flush procedures require different types and amounts of flush solutions. The most commonly used flush solutions are saline and/or heparin lock solution. The type of flush solution and amount vary depending on the specific type of catheter. Flush solution volumes between 5 and 10 ml are most common but can range from 1 ml to 20 ml.

With respect to flush procedures, an IV line refers to a system that can include a VAD, a tubing set with clamp and a VAD connector as a termination. Common types of VAD connectors are covered by pierceable septums or pre-slit septums made of rubber or another elastomeric material, which permits insertion of a sharp needle cannula in order to infuse fluids into or to withdraw fluids from the catheter. Upon withdrawal of the needle cannula, the septum seals itself. Ports having pre-slit septums are used with blunt plastic cannula or the frusto-conically shaped tip of a syringe barrel. The flush syringe tip or the blunt plastic cannula (which is usually attached to a syringe) is gently pushed through the pre-slit septum to establish fluid communication.

IV valves, another type of VAD connector that does not require a needle having a sharp tip, are activated by the frusto-conically shaped tip of a syringe barrel to allow fluid communication between the interior of the flush syringe and the catheter. These valves may contain features for delivering fluid from a storage compartment in the valve to the catheter, and are referred to in the art as positive displacement valves.

Bacteria and other microorganisms may gain entry into a patient's vascular system from access hubs and ports/valves upon connection to the VAD to deliver the fluid or pharmaceutical. Each access hub (or port/valve or connection) is associated with some risk of transmitting a catheter related bloodstream infection (CRBSI), which can be costly and potentially lethal.

Throughout the sequence of procedures associated with the transmission of a microorganism that can cause a CRBSI, there are many risks of contact or contamination. Contamination can occur during drug mixing, attachment of a cannula, and insertion into the access hub. Because the procedure to connect to a VAD is so common and simple, the risk associated with entry into a patient's vascular system has often been overlooked. Presently, the risk to hospitals and patients is a substantial function of the diligence of the clinician performing the connection, and this diligence is largely uncontrollable.

Current “recommended practice” for aseptic IV line maintenance and IV drug delivery practices require adherence to a stepwise process referred to as “SASH.” During the first step of the process, the clinician cleans/disinfects (generally with an alcohol swab) the VAD connector. Second, a syringe containing saline is used to flush the IV line or catheter (Saline flush), and then the VAD connector is disinfected a second time. Third, the fluid or pharmaceutical therapy is administered through the IV line or catheter (Administer therapy), the VAD connector is disinfected a third time, followed by a second Saline flush step. The final step, which is dependent upon the patient's need and institutional policy, is a final disinfection of the VAD connector followed by a Heparin lock step, where a small amount of heparin is injected into the IV line or catheter to prevent the formation of thrombi or blood clots. At the conclusion of this tedious stepwise process, the inlet port of the VAD connector is left exposed to the environment. This “recommended practice” requires disinfecting the VAD connector after each step, and makes IV line maintenance a very burdensome and time consuming process. Because the process is so cumbersome, clinicians very rarely implement this “recommended practice” in its entirety, and, thus, patients are exposed to the risk of contracting CRBSIs. Microorganisms populate exposed connector inlet surfaces, and, when the “recommended practice” is not adhered to, the microorganisms can enter the IV line during flushing. Furthermore, blood reflux into the IV line or catheter can cause clot formation inside the lines, and microorganisms from the connector inlet surfaces can colonize blood clots inside the lines and infect the patients during flushing.

One product currently available that aims to combat the problems associated with contaminated VAD connectors is the SwabCap® from ICU Medical, Inc. Such capping devices disinfect a VAD connector by covering the connector and protecting it from touch and airborne contamination after the cap has been applied. As the capping device is twisted onto VAD connector, a foam pad inside the cap is compressed, releasing the isopropyl alcohol that bathes and passively disinfects the top and threads of the VAD connector while the cap is in place. Friction between the capping device and VAD connector is essential to ensure proper swabbing and disinfecting as the twisting action helps focus the alcohol on the targeted areas. However, for several reasons, such capping devices fall short of accomplishing the desired goal of effectively cleaning and disinfecting the VAD connector. First, the caps do not always engage the threads on the catheter hub, so that friction during swabbing may be inefficient. Additionally, the caps are small, and thus, may result in touch contamination when they are being removed. Despite the fact that the caps may be distinctively colored (e.g., bright orange) so that compliance can be visually confirmed, because such caps are physically separate items, only the most diligent clinician will utilize the cap after every step of the flush process. Thus, these types of caps do not ensure compliance with aseptic technique.

Substantial morbid and mortal risk is associated with a number of routine procedures defined primarily by the uncontrollable diligence of the clinician administering the therapy. Unfortunately, the result is that a substantial degree of unnecessary risk and injury, in the form of CRBSIs, to patients occurs due to low compliance rates. Accordingly, there is a need for syringes that promote compliance with aseptic techniques by eliminating the additional swabbing and disinfecting steps.

### SUMMARY

A first aspect of the present disclosure relates to a flush syringe and integrated swab assembly comprising a sidewall defining a barrel having an inside surface defining a chamber configured to retain a fluid, an open proximal end, a distal end including a distal wall with a tip extending distally therefrom having a passageway therethrough in fluid communication with the chamber; a plunger rod comprising a proximal end and a distal end including a stopper slidably positioned in the chamber and configured to draw fluid into an drive fluid out of the chamber by movement of the stopper relative to the chamber, the plunger rod extending outwardly from the open proximal end of the chamber; an integral collar extending from the distal wall of the barrel, surrounding the distal tip, and having a collar distal end and a collar proximal end, the integral collar including at least one sidewall having an inside surface including an inner thread thereon defining a Luer lock area and configured to connect to the hub of a vascular access device connector, the at least one sidewall further having an outside surface including an outer thread thereon configured for connection to a removable cap that when connected to the integral collar covers the integral collar to protect the distal tip 106 and the collar from contamination; and an integral swab comprising a porous hollow mass threadably connected to the inner thread of the integral collar, the porous hollow mass entrained with a disinfectant or an antimicrobial agent, wherein the removable cap is further configured to cover the integral swab.

### BRIEF DESCRIPTION OF THE DRAWINGS

FIG. 1A is a perspective view of a flush syringe and integrated swab assembly according to a first embodiment of the present disclosure;

FIG. 1B is a partial side cross-section view of the flush syringe and integrated swab assembly of FIG. 1A;

FIG. 1C is a perspective view of the flush syringe and integrated swab assembly shown in FIG. 1A initially engaged with a vascular access device connector;

FIG. 1D is a partial side cross-section view showing the flush syringe and integrated swab assembly shown in FIG. 1A connected to a vascular access device connector;

FIG. 1E is a perspective view of the flush syringe and integrated swab assembly shown in FIG. 1A further engaged with the vascular access device connector with the vascular access device connector after the two components have been pushed together and threadably engaged;

FIG. 1F is a partial side cross-section view showing the flush syringe and integrated swab assembly shown in FIG. 1A further engaged with the vascular access device connector with the vascular access device connector after the two components have been pushed together and threadably engaged;

FIG. 1G is a perspective view of a flush syringe that can be used with one or more embodiments of the flush syringe and integrated swab assembly described herein;

FIG. 2A is a proximal view of the distal end of the flush syringe and integrated swab assembly according to a first embodiment of the present disclosure;

FIG. 2B is a side elevation view of the flush syringe and integrated swab assembly shown in FIG. 2A;

FIG. 3A is a perspective view of a flush syringe and integrated swab assembly according to a second embodiment of the present disclosure;

FIG. 3B is a partial side cross-section view of the flush syringe and integrated swab assembly of FIG. 3A;

FIG. 3C is a perspective view of the flush syringe and integrated swab assembly shown in FIG. 3A initially engaged with a vascular access device connector;

FIG. 3D is a partial side cross-section view showing the flush syringe and integrated swab assembly shown in FIG. 3A connected to a vascular access device connector;

FIG. 3E is a perspective view of the flush syringe and integrated swab assembly shown in FIG. 3A further engaged with the vascular access device connector with the vascular access device connector after the two components have been pushed together and threadably engaged;

FIG. 3F is a partial side cross-section view showing the flush syringe and integrated swab assembly shown in FIG. 3A further engaged with the vascular access device connector with the vascular access device connector after the two components have been pushed together and threadably engaged;

FIG. 3G is a perspective view of the swab of the flush syringe and integrated swab assembly shown in FIG. 3A;

FIG. 3H is a is perspective view of the swab attached to the distal end of the flush syringe and integrated swab assembly shown in FIG. 3A;

FIG. 3I is a side elevation view of the swab attached to the distal end of the flush syringe and integrated swab assembly shown in FIG. 3A;

FIG. 4A is a perspective view of a flush syringe and integrated swab assembly according to a third embodiment of the present disclosure;

FIG. 4B is a partial side cross-section view of another embodiment of a flush syringe and integrated swab assembly;

FIG. 4C is a perspective view of the flush syringe and integrated swab assembly shown in FIG. 4A initially engaged with a vascular access device connector;

FIG. 4D is a partial side cross-section view showing the flush syringe and integrated swab assembly shown in FIG. 4B initially engaged with a vascular access device connector;

FIG. 4E is a perspective view of the collar of the flush syringe and integrated swab assembly shown in FIG. 4A with the collar in the open position;

FIG. 4F is a side elevation view of an alternative configuration of a collar of the flush syringe and integrated swab assembly shown in FIG. 4B with the collar in the open position;

FIG. 4G is a perspective view of the distal end of the alternative configuration shown in FIG. 4B;

FIG. 4H is a side elevation view of the alternative configuration shown in FIG. 4B;

FIG. 5A is a perspective view of a flush syringe and integrated swab assembly according to a fourth embodiment of the present disclosure;

FIG. 5B is a partial side cross-section view of the flush syringe and integrated swab assembly of FIG. 5A;

FIG. 5C is a perspective view of the flush syringe and integrated swab assembly shown in FIG. 5A initially engaged with a vascular access device connector;

FIG. 5D is a partial side cross-section view showing the flush syringe and integrated swab assembly shown in FIG. 5A connected to a vascular access device connector;

FIG. 5E is a perspective view of the flush syringe and integrated swab assembly shown in FIG. 5A further engaged with the vascular access device connector with the vascular access device connector after the two components have been pushed together and threadably engaged;

FIG. 5F is a partial side cross-section view showing the flush syringe and integrated swab assembly shown in FIG. 5A further engaged with the vascular access device connector with the vascular access device connector after the two components have been pushed together and threadably engaged;

FIG. 5G is a perspective view of a distal end of another embodiment of a flush syringe and integrated swab assembly;

FIG. 5H is a side elevation view of the distal end of the flush syringe and integrated swab assembly of FIG. 5G;

FIG. 6A is a perspective view of a flush syringe and integrated swab assembly according to a fifth embodiment of the present disclosure;

FIG. 6B is a partial side cross-section view of another embodiment of a flush syringe and integrated swab assembly;

FIG. 6C is a perspective view of the flush syringe and integrated swab assembly shown in FIG. 6B initially engaged with a vascular access device connector;

FIG. 6D is a partial side cross-section view showing the flush syringe and integrated swab assembly shown in FIG. 6B connected to a vascular access device connector;

FIG. 6E is a perspective view of the flush syringe and integrated swab assembly shown in FIG. 6A connected to the vascular access device connector with the vascular access device connector after the two components have been pushed together and threadably engaged;

FIG. 6F is a partial side cross-section view showing another embodiment of a flush syringe and integrated swab assembly shown in engaged with the vascular access device connector with the vascular access device connector after the two components have been pushed together and threadably engaged;

FIG. 6G is perspective view of a distal end of an alternative configuration of the flush syringe and integrated swab assembly; and

FIG. 6H is a side elevation view of the distal end of the flush syringe and integrated swab assembly of FIG. 6G.

### DETAILED DESCRIPTION

Embodiments of the disclosure pertain to flush syringe and integrated swab assemblies. With respect to terms used in this disclosure, the following definitions are provided.

Reference to “flush syringe assembly” includes syringes that are indicated for use in the flushing of VADs. The practice of flushing ensures and maintains catheter patency and helps prevent the mixing of incompatible pharmaceuticals.

As used herein, the use of “a,” “an,” and “the” includes the singular and plural.

As used herein, the term “catheter related bloodstream infection” or “CRBSI” refers to any infection that results from the presence of a catheter or IV line.

As used herein, the term “microorganism” refers to a microbe or organism that is unicellular or lives in a colony of cellular organisms. Microorganisms are very diverse; they include, but are not limited to bacteria, fungi, archaea, and protozoans. Microorganisms are often the cause of CRBSIs. The most common microorganisms associated with CRBSIs include, but are not limited to, Staphylococcus aureus and epidermis, Enterococcus faecalis, Escherichia coli, Pseudomonas aeruginosa, and Candida albicans.

As used herein, the terms “antimicrobial agent” or “antimicrobial” refers to substances that kill or inhibit the growth of microorganisms such as bacteria, fungi, archaea, or protozoans. Antimicrobial agents either kill microbes, or prevent the growth of microbes.

As used herein, the term “disinfectant” refers to antimicrobial substances that are used on non-living objects or outside the body, e.g., on the skin.

In one or more embodiments, disinfectants or antimicrobial agent include, but are not limited to, ethanol, 2-propanol, butanol, methylparaben, ethylparaben, propylparaben, propyl gallate, butylated hydroxyanisole (BHA), butylated hydroxytoluene, t-butyl-hydroquinone, chloroxylenol, chlorohexidine, dichlorobenzyl alcohol, dehydroacetic acid, hexetidine, triclosan, hydrogen peroxide, colloidal silver, and mixtures thereof.

As used herein, the term “absorbent material” refers to a material having capacity or tendency to absorb or soak up another substance. In one or more embodiments, the absorbent material has a tendency to absorb a disinfectant or antimicrobial. Absorbent materials may include sponges, absorbent cottons, other absorbent fabrics, and synthetic polymer matrices.

As used herein, the term “Luer connector” refers to a connection collar that is the standard way of attaching syringes, catheters, hubbed needles, IV tubes, etc. to each other. The Luer connector consists of male and female interlocking tubes, slightly tapered to hold together better with even just a simple pressure/twist fit. Luer connectors can optionally include an additional outer rim of threading, allowing them to be more secure. The Luer connector male end is generally associated with a flush syringe and can interlock and connect to the female end located on the VAD. A Luer connector comprises a distal end, a proximal end, an irregularly shaped outer wall, a profiled center passageway for fluid communication from the chamber of the barrel of a syringe to the hub of a VAD. A Luer connector also has a distal end channel that releasably attaches the Luer connector to the hub of a VAD, and a proximal end channel that releasably attaches the Luer connector to the barrel of a syringe.

According to one or more embodiments described herein, a flush syringe and integrated swab assembly is provided. In some embodiments, the flush syringe has a swab in the form of a disinfectant-containing (or loaded or infused) porous material surrounding the flush syringe tip. In some embodiments, the swab has an opening and/or slits on a distal “top” portion to allow a needleless connector to penetrate the swap and to connect to the flush syringe tip. The swab will deform in way so as to create sufficient friction and scrubbing between the connector and swab and to release the disinfectant as it gets compressed to disinfect the connector surfaces. In one or more embodiments, the connector is needleless. Following full engagement of the flush syringe and the connector, the flush syringe content can be administered without disconnecting the swab that is integrated with the flush syringe. Embodiments advantageously increased compliance of cleaning of connectors prior to connection. The practitioner is not required to open a separate cleaning element such as a wipe or a separate swab. By simply connecting through the integrated swab surrounding the distal tip of the flush syringe, the connector is cleaned during the connection process.

Various embodiments and variants are described in this disclosure. In some embodiments, the flush syringe and integrated swab assembly has an extended barrel section that houses the swab. For example, a molded tip cap or a seal can be used to close off the area. In some embodiments, the swab is located at a distal end of the syringe without lateral support. For example, the distal tip cap will thread onto the barrel itself at the top portion of the barrel near the swab, wherein the barrel will have threads near the end that is by the swab.

In one or more embodiments, the swab can assume a variety of shapes including, but not limited to a cylindrical or hexagonal outer surfaces. The swab can be made of one piece or multiple segments. In some embodiments, the swab has additional surface features to enhance scrubbing and disinfection, such as in embodiment scrubbing bristles.

Embodiments provide a mechanism to allow surface contact and through access to the needleless (need free) connector can assume a range of options. Some embodiments provide pre-cut slits in the swab that can open upon pressure when the connector is pushed together with the swab. In some embodiments, the swab, which can be in the form of a foam element such as a sponge, splits and two halves detach completely to give way to the connector, allowing the connection to be completed while cleaning the connector. In some embodiments, two paddles protrude outwardly from the flush syringe barrel surface. When pushed in against the barrel, the system opens at the distal end to allow the connector to be pushed through and connected to the flush syringe tip.

An additional design feature is minimizing mixing of saline with the disinfectant. For example, in some embodiments, a feature is provided to physically keep the disinfectant away from the flush syringe tip.

Advantageously, embodiments allow for the disinfection of a wide variety of needle-free connectors. One or more embodiments allow the air to be expelled from the flush syringe prior to connecting with and disinfecting the needle-free connector. Some embodiments also have an incorporated window to allow for visualizing the flush syringe tip to check on expelling of air bubbles. In some embodiments, the porosity of the swab can vary through different regions of the swab (e.g., a radial gradient or axial gradient) to control the absorbance and release of the disinfectant. Additionally, the distal surface can include additional surface features for improved scrubbing.

Advantageously, embodiments ensure compliance with clinical best practices which requires that clinicians disinfect the needleless connector. Embodiments described in this disclosure concepts provides enforced compliance, which ultimately reduces the chances of hospital acquired infections. The practitioner will have to open fewer packages, does not have to carry alcohol swabs, and will be able to combine two steps into one, thus simplifying workflow.

In each of the embodiments described below, the common components of the flush syringe and integrated swab assembly are similarly numbered for each embodiment. Thus, the sidewall 101 defining the barrel of the flush syringe and integrated swab assembly.

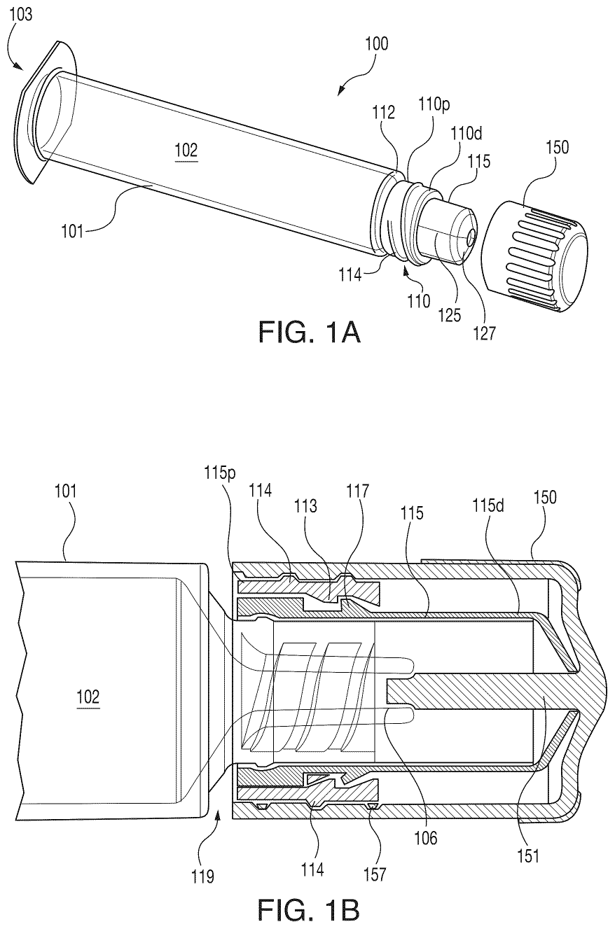

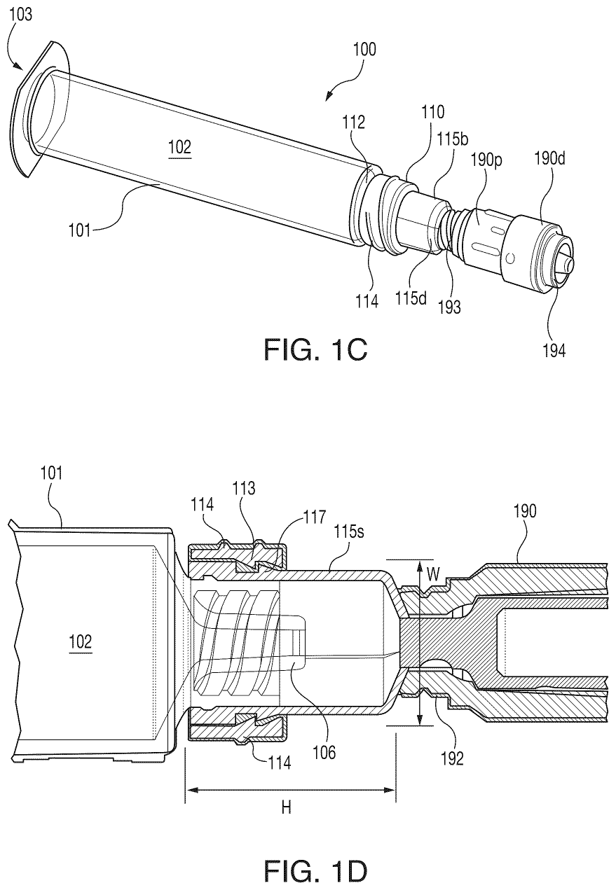

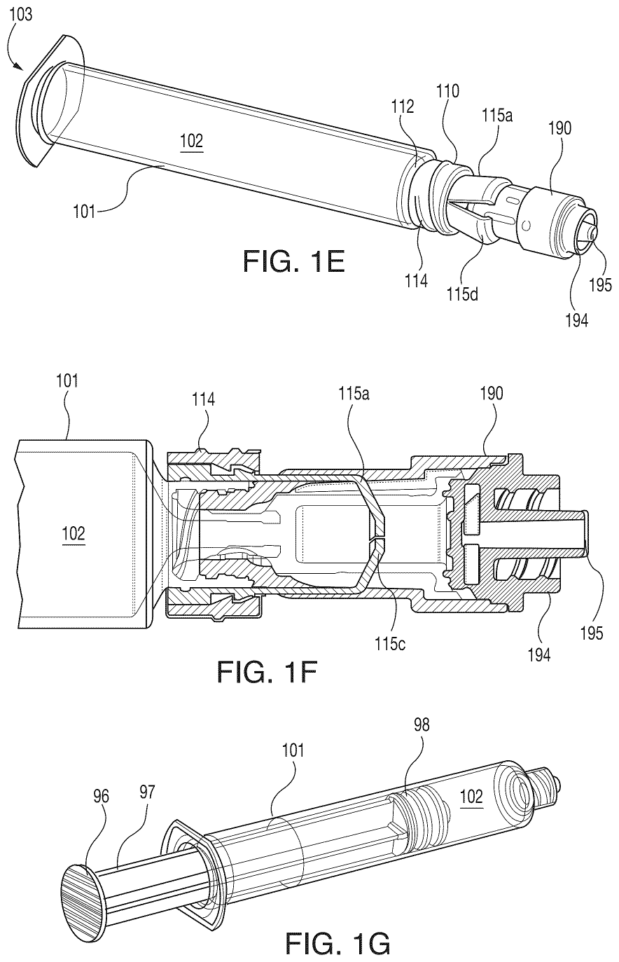

Referring now to FIGS. 1A-F, a first embodiment of a flush syringe and integrated swab assembly 100 comprises a sidewall 101 defining a barrel having an inside surface defining a chamber 102 configured to retain a fluid, an open proximal end 103, a distal end 110d including a distal wall 112 with a distal tip 106 extending distally therefrom having a passageway 107 therethrough in fluid communication with the chamber 102. FIG. 1G shows a flush syringe that can be used in accordance with the embodiments of the flush syringe and integrated swab assembly described herein. The flush syringe comprises the sidewall 101 defining the chamber 102 and includes a plunger rod 97 comprising a proximal end having a thumb press 96 on the proximal end and a distal end including a stopper 98 slidably positioned in the chamber 102 and configured to draw fluid into and drive fluid out of the chamber 102 by movement of the stopper relative to the chamber 102. The plunger rod extends outwardly from the open proximal end 103 of the chamber.

Referring now to FIG. 1A, an integral collar 110 extends from the distal wall 112 of the barrel, surrounding the distal tip 106. The integral collar 110 has a collar distal end 110d and a collar proximal end 110p, and the integral collar 110 includes at least one sidewall having an inside surface including an inner thread 113 thereon defining a Luer lock area and configured to connect to the hub of a vascular access device connector. The at least one sidewall further has an outside surface including an outer thread 114 thereon configured for connection to a removable cap 150 that, when connected to the integral collar 110 covers the integral collar 110 to protect the distal tip 106 and the integral collar 110 from contamination.

Still referring to FIG. 1A, the integrated flush syringe and swab assembly further includes an integral swab 115 comprising a porous hollow mass threadably connected to the inner thread 113 of the integral collar 110, the porous hollow mass that forms the integral swab 115 is entrained with a disinfectant or an antimicrobial agent. The removable cap 150 is further configured to cover the integral swab 115 to keep the integral swab moist with disinfectant or antimicrobial. The integral swab 115 includes swab threads 117 that engage internal cap thread 157 on an inner surface of the removable cap 150.

As used herein, “integral” and “integrated” with respect to the integral swab or integrated swab refers to the integral swab that is formed as a unit with the integral collar 110 of the flush syringe of the integrated flush syringe and swab assembly. This means that the integral collar connected to or assembled to the integral collar 110 and is not a separate or separated component that is attached prior to use. The flush syringe and integrated swab assembly 100 is packaged as a unitary assembly and ready for use, where the swab is not a separate part that must be connected to the integral collar.

In the embodiment shown, the porous hollow mass that forms the integral swab 115 may be any suitable porous material such as a porous polymer such as a cast porous urethane material or a porous sponge. As shown in FIGS. 1A-F, the integral swab 115 can be in the form of a hollow cylinder having a proximal end 115p with the threads 117 closer to the proximal end 115p of the integral swab 115, and the porous hollow cylinder is threadably connected to the inner thread 113 of the integral collar 110 so that the proximal end 115p of the porous hollow cylinder that forms the integral swab 115 is spaced from the collar proximal end 110p, providing a visible window 119 between the proximal end 110p of the hollow cylinder that forms the integral swab 115 and the proximal end 110p of the integral collar 110 extending from the chamber 102.

Referring now to FIG. 1C, which is a perspective view of the flush syringe and integrated swab assembly 100 shown in FIG. 1A showing the initially engaged with a vascular access device connector 190 having a proximal end 190p included a connection thread 193 configured to connect to the inner thread 113 of the flush syringe and integrated swab assembly 100 and a distal end 190d having a connection 196 configured to connect to a medical device such as a vascular access device (e.g., a catheter). The vascular access device connector 190 includes a fluid passageway 195 that, when the vascular access device connector 190 is connected with the flush syringe and integrated swab assembly 100, is in fluid communication with the chamber 102 so that the fluid in the chamber can be utilized to flush a medical device. In use, a clinician or medical practitioner aligns the vascular access device connector 190 with the integral swab 115 and pushes the two components together as shown in FIGS. 1C and 1D.

FIGS. 1E and 1D show views of the flush syringe and integrated swab assembly 100 further engaged with the vascular access device connector 190 with the vascular access device connector after the two components have been further pushed together and rotated to engage the connection thread 193 with the inner thread 113 of the flush syringe and integrated swab assembly 100. This results in threadable engagement of the two components to secure the flush syringe and integrated swab assembly 100 and the vascular access device connector 190 to each other.

The action of pushing the two components together and rotating the vascular access device connector 190 with respect to the integral swab 115 extending from the flush syringe and integrated swab assembly 100 causes the distal face and peripheral surface (the connection thread 113) to be scrubbed and sterilized the distal end 190d of the vascular access device connector 190. Because the integral swab 115 is integrated with the flush syringe and integrated swab assembly 100, the practitioner does not have an option to skip the cleaning and disinfection of the vascular access device connector 190, which ensures a higher degree of compliance compared to existing solutions that require separate items or separate connection of a swab to the end of a flush syringe.

Still referring to FIGS. 1A-F, and in particular, FIGS. 1A and 1D, the integral swab 115 comprising the hollow cylinder has a height dimension H and a cross-sectional width dimension W. There is at least one slit 125 through the cross-sectional width W defining at least two separable projections 115a and 115c that are configured to separate upon insertion of a vascular access device connector and connection to the inner thread of the integral collar 110. In the embodiment shown, there is a second slit 127 and there are four separable projections 115a, 115b, 115c and 115d that are configured to separate upon insertion of a vascular access device connector and connection to the inner thread of the integral collar 110. The at least two separable projections 115a and 115c are configured to contact the vascular access device connector on an outer peripheral surface and a distal surface of the vascular access device connector.

The at least two separable projections 115a and 115c provide two separate halves of the integral swab 115 divided by the at least one slit 125. When there is a second slit 127, each of the four separable projections 115a, 115b, 115c, and 115d provide four separate quarters of the integral swab 115 divided by the two slits. Other configurations are within the scope of the disclosure. For example, slits can be provided so that there would be any number of separable projections, for example, three, five, six, seven, eight or more separable projections that are configured to contact the vascular access device connector on an outer peripheral surface and a distal surface of the vascular access device connector 190 upon insertion of the vascular access device connector 190 and connection to the inner thread 113 of the integral collar 110.

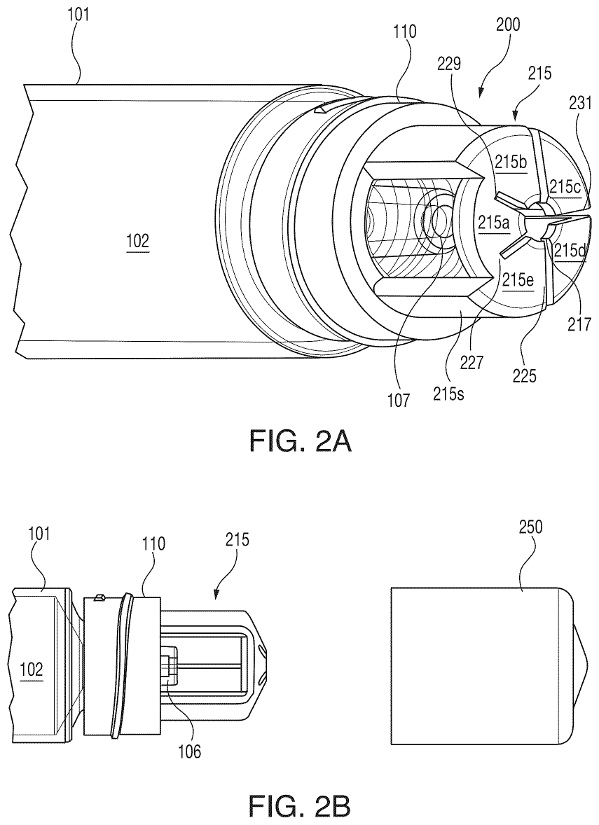

FIGS. 2A and 2B show an alternate embodiment of a flush syringe and integrated swab assembly 200, where the flush syringe components are the same and comprise the sidewall 101, integral collar 110 and the chamber as described with respect to FIGS. 1A-G. The integral swab 215 comprises a hollow cylinder which has a height dimension H and a cross-sectional width dimension W as shown in the previously described embodiment. The integral swab 205 shown in FIGS. 2A and 2B comprises a first slit 225 dividing the integral swab 205 into a first separable projection 215a and a second separable projection 215c that are configured to contact the vascular access device connector on an outer peripheral surface and a distal surface of the vascular access device connector 190 upon insertion of the vascular access device connector 190 and connection to the inner thread 113 of the integral collar 110. The integral swab 205 further comprises three smaller slits 227, 229 and 231, further dividing the integrated swab into additional separable projections 215b, 215d and 215e. There is a central opening 217 in the crown formed by the separable projections to allow the vascular access device connector to be connected to the integrated flush syringe and swab assembly 200.

The separable projections 115a-d shown in FIGS. 1A-F and 215a-e shown in FIGS. 2A-B provide what may be referred to as a crown portion of the integral swab 115 and 215. Thus, the integral swab 115 comprises a crown portion and a skirt 115s extending in a proximal direction from crown portion formed by the separable projections. Similarly, in FIGS. 2A-B, the integral swab 215 comprises a crown portion and a skirt 215s extending in a proximal direction from crown portion formed by the separable projections. In some embodiments, the at least one slit extends through the skirt 115S or 215S, as shown in FIG. 2A. In FIG. 2A, the two slits 125 and 127 extend through the skirt 115S. A removable cap 250 is removably attached to cover the integral swab 205 similar to the embodiment shown in FIGS. 1A-F.

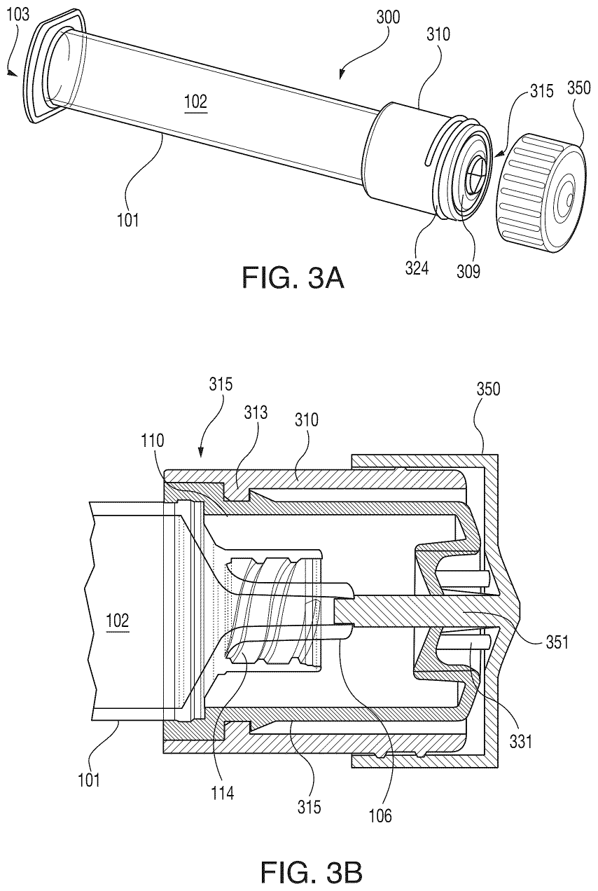

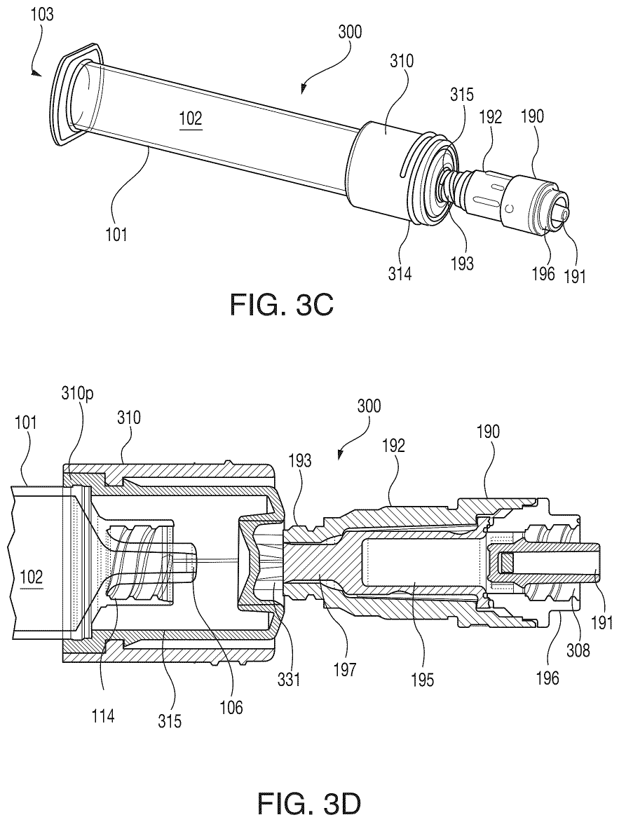

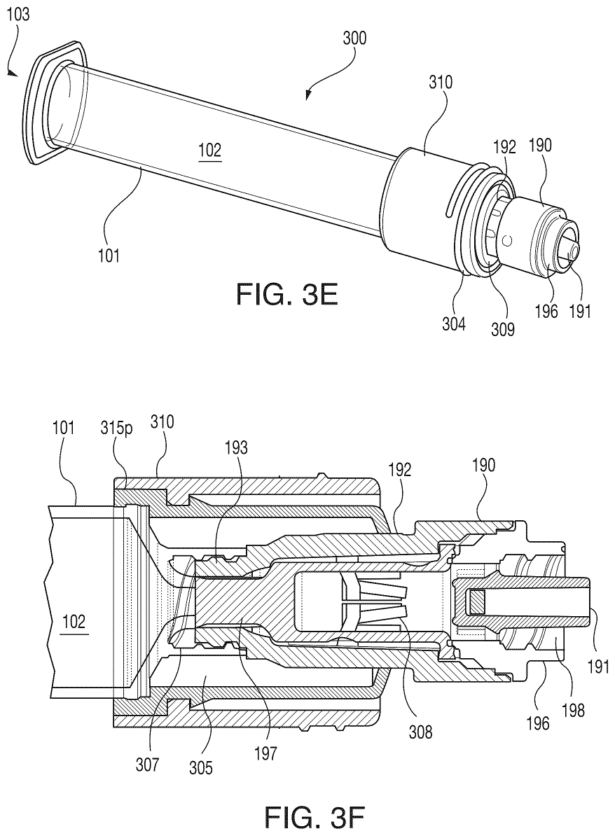

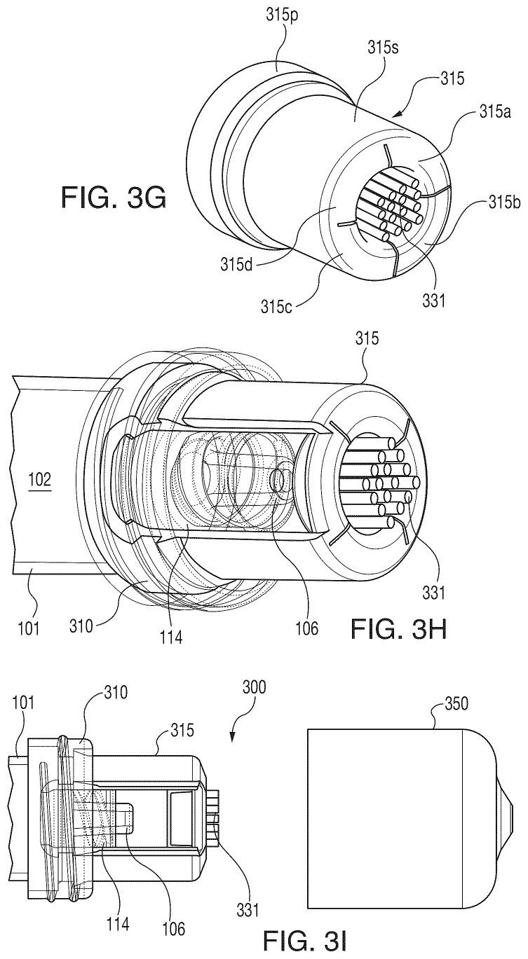

Referring now to FIGS. 3A-F, another embodiment of a flush syringe and integrated swab assembly 300 is shown, where the flush syringe components are the same and comprise the sidewall 101, integral collar 110 and the chamber as described with respect to FIGS. 1A-G. In the embodiment shown there is a crown portion comprised of four separable projections 315a, 315b, 315c and 315d, similar to the embodiment shown in FIGS. 1A-F, where the four separable projections 315a-d are provided by slits through the integral swab 315 comprising a hollow cylinder having a height dimension H and a cross-sectional width dimension W. In the embodiment shown in FIGS. 3AG, the crown portion further comprises a plurality of bristles 308. The plurality of bristles 308 is arranged in a circular pattern to provide additional scrubbing and disinfection ability.

The flush syringe and integrated swab assembly according to claim 6, wherein the crown portion further comprises a plurality of bristles 331 extending in a distal direction from the crown region that is made up of the four separable projections 315a, 315b, 315c and 315d. A skirt 315s extends in a proximal direction from the crown region that is made up of the four separable projections 315a, 315b, 315c and 315d. Similar to the previously described embodiments, the integral swab 315 can be connected via a thread on a proximal portion 315p of the integral swab 315. However, in the embodiment shown in FIGS. 3A-G, the proximal portion 315p of the integral swab 315 comprises a flare end that attaches to the sidewall 101 of syringe and an auxiliary collar 310 having an external thread 324 is also secured to the sidewall 101 of the flush syringe. Thus, the proximal portion 315p of the integral swab 315 is secured between the auxiliary collar 310 and the sidewall 101. The auxiliary collar 310 shown includes an internal thread 313, and the integral swab 315 can be threadably attached to the auxiliary collar 310. A removable cap 350 is removably attached to the auxiliary collar 310 by the external thread 324 on the auxiliary collar 315 and internal cap thread 357 on the removable cap 350.

The crown portion made up of the four separable projections 315a, 315b, 315c and 315d and the plurality of bristles are configured to contact an end surface of a vascular access device connector 190 to clean and disinfect the connector.

In some embodiments, the removable cap (see removable cap 150 and removable cap 350 comprises a central finger 351 as shown in FIG. 1A-F or a central finger 351 as shown in FIGS. 3A-G extending in a proximal direction and into the passageway 108 of the distal tip 106 to prevent entry of fluid into the chamber. that extends through the plurality of bristles and into the passageway of the distal tip to prevent entry of fluid into the chamber 102. In FIGS. 3A-G, the central finger 351 extends through the plurality of bristles 331. FIGS. 3H and 3I show alternate view of the integrated flush syringe and swab assembly 300.

Connection and simultaneous cleaning and disinfection of the vascular access device connector 190 to the integrated flush syringe and swab assembly 300 proceeds similarly as with the previously described embodiments. FIGS. 3C and 3D show initial connection of the two components by aligning the vascular access device connector 190 with the integral swab 315 by pushing the elements together. Then, as shown in FIGS. 3E and 3F, the vascular access device connector 190 and the integrated flush syringe and swab assembly 300 are rotated with respect to each other to threadably connect the components and simultaneously clean and disinfect the proximal end 190p of the vascular access device connector 190.

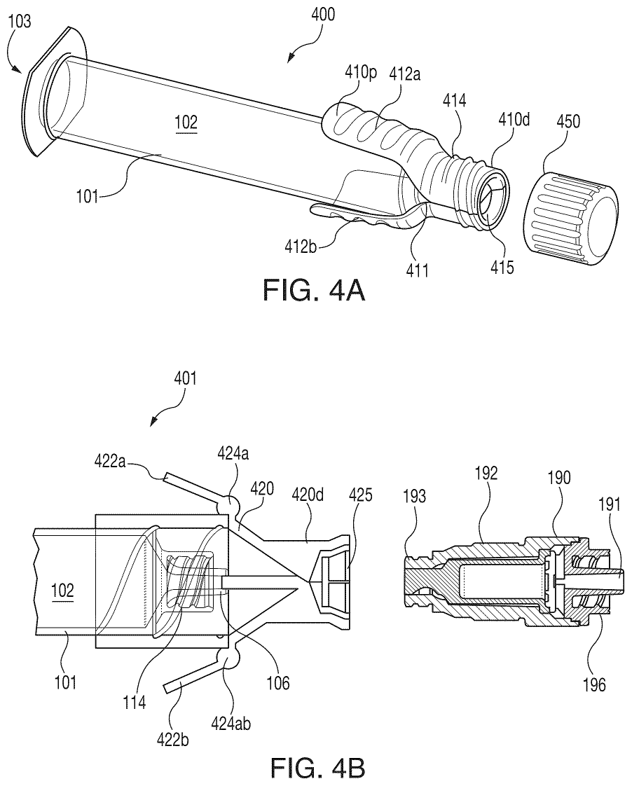

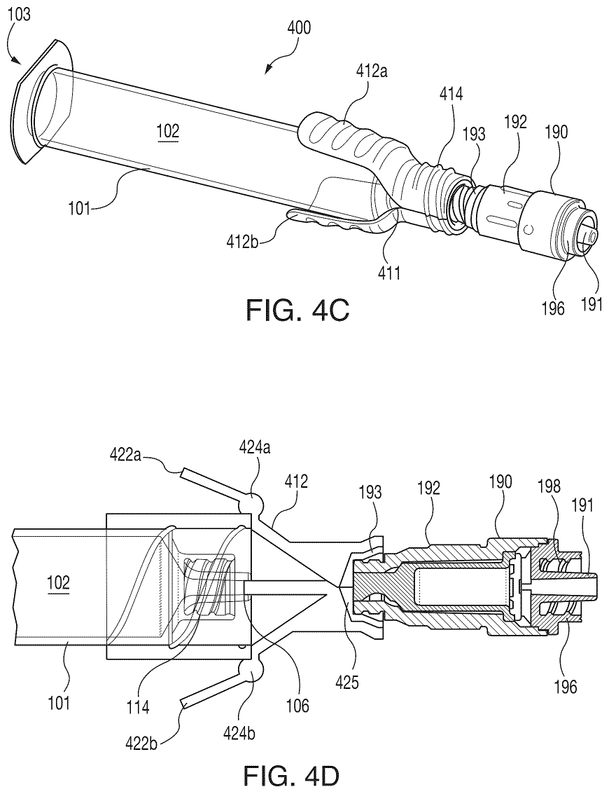

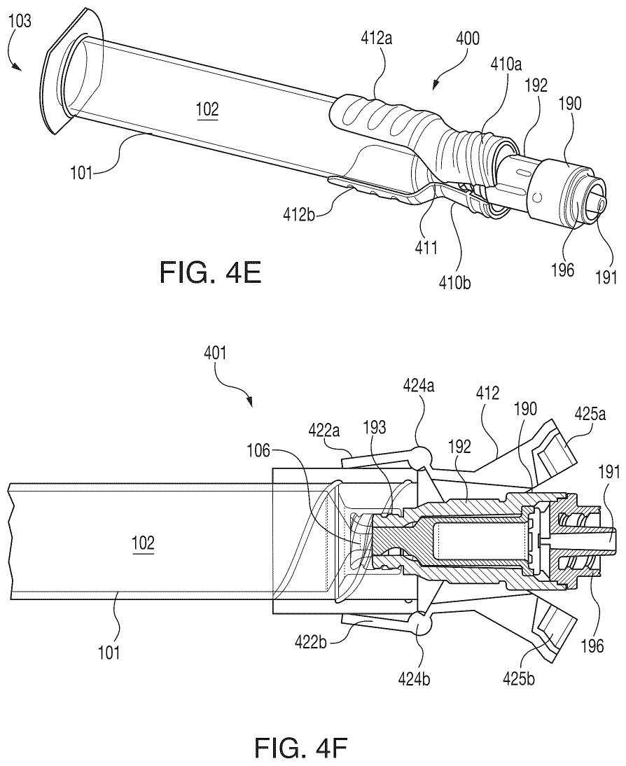

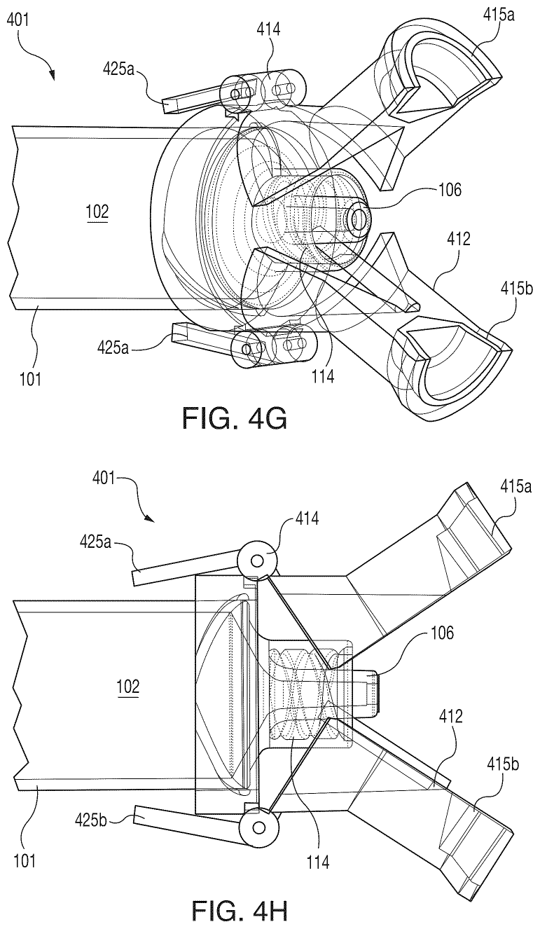

Referring to FIGS. 4A-H, embodiments of a flush syringe and integrated swab assembly 400, 401 are shown including an auxiliary collar with a hinge. The auxiliary collar is attached to the sidewall of the flush syringe. In FIGS. 4A, 4C and 4E, the integrated flush syringe and integrated swab assembly 400 comprises an auxiliary collar 410 attached to the sidewall 101 and including a hinge, shown as a living hinge 411 and paddles 412a, 412b extending from the living hinge 411 in a proximal direction. The distal end 410d of the auxiliary collar 410 is divided into a first section 410a and a second section 410b. The paddles 412a, 412b are configured to open the distal end 410d of the auxiliary collar 410 by squeezing the paddles 412a, 412b to expose the distal tip 106 of the flush syringe. An integral swab 415 is contained within the auxiliary collar 410, and the auxiliary collar 410 has an external thread 414 to allow a removable cap 450 to be threadably connected to the auxiliary collar 410.

An integral swab 415 is mounted within the auxiliary collar 410 at the proximal end 410p. The integral swab 415 in one or more embodiments is similar to the integral swab 115 described with respect to FIGS. 1A-F. The integral swab 415 of some embodiments comprises a porous hollow mass threadably connected to the distal portion of the auxiliary collar 410. The porous hollow mass that forms the integral swab 415 is entrained with a disinfectant or an antimicrobial agent.

Similar to the integral swab 115, the integral swab can be in the form of a hollow cylinder having a proximal end with the threads closer to the proximal end of the integral swab 415, and the porous hollow cylinder is threadably connected to an inner thread of the auxiliary collar 410.

The integral swab 415 can be similar to the integral swab 115 comprising the hollow cylinder has a height dimension H and a cross-sectional width dimension W. There can at least one slit through the cross-sectional width W defining at least two separable projections that are configured to separate upon insertion of a vascular access device connector and connection to the inner thread of the auxiliary collar 410. There can also be a second slit to provide four separable projections that are configured to separate upon insertion of a vascular access device connector. The at least two separable projections are configured to contact the vascular access device connector on an outer peripheral surface and a distal surface of the vascular access device connector.

The at least two separable projections provide two separate halves of the integral swab 415 divided by the at least one slit. When there is a second slit, each of the four separable projections provide four separate quarters of the integral swab 415 divided by the two slits. Other configurations are within the scope of the disclosure. For example, slits can be provided so that there would be any number of separable projections, for example, three, five, six, seven, eight or more separable projections that are configured to contact the vascular access device connector on an outer peripheral surface and a distal surface of the vascular access device connector 190 upon insertion of the vascular access device connector 190 and connection to the auxiliary collar 410.

Referring to FIGS. 4C and 4E, in use, a practitioner squeezes the paddles 412a and 412B, and the first section 410a and the second section 410b pivot about the living hinge 411, causing the auxiliary collar 410 to open. The vascular access device connector 190 is then aligned with the integral collar 110 and pushed together as shown in FIG. 4C. The practitioner then rotates the vascular access device connector 190 and the integrated flush syringe and swab assembly 400 with respect to each other to threadably connect the components and simultaneously clean and disinfect the proximal end 190p of the vascular access device connector 190.

FIGS. 4B-D and F-H show a second embodiment of a flush syringe and integrated swab assembly 401 including an auxiliary collar 420. The auxiliary collar 420 comprises distal end 420d that is divided in a first section 420a and a second section 420b. The first section 420a comprises a first hinge 424a and the second section 420b comprises a second hinge 424b. The first section 420a and the second section 420b are configured to respectively pivot about the first hinge 424a and the second hinge 424b from a closed position as shown in FIG. 4B and FIG. 4D to an open position a as shown in FIGS. 4F-H to expose the distal tip 106 of the flush syringe.

The flush syringe and integrated swab assembly 401 shown further includes a first lever 425a associated with the first section 420a and the first hinge 424a and a second lever 425b associated with the second section 420b. The first lever 425a and the second lever 425b are configured to respectively rotate the first section 420a and the second section 420b from the closed position to an open position to expose the distal tip 160 of the flush syringe.

Referring now to FIG. 4D, a practitioner aligns the vascular access device connector 190 with the auxiliary collar 420 and pushes the two components together and rotates the vascular access device connector 190 with respect to the integrated flush syringe and swab assembly 401 so that the swab 425 contacts the distal portion of the vascular access device connector to clean and disinfect the connector. Then, the practitioner opens the first section 420a and the second section 420b by squeezing the first lever 425a and the second lever 425b to expose the distal tip of the flush syringe. The practitioner then connects the vascular access device connector by engaging the thread on each component and twisting to lock the vascular access device connector to the integrated flush syringe and swab assembly 401. FIGS. 4G and 4H show alternate views of the auxiliary collar 420 in the open position and ready to receive the vascular access device connector 190.

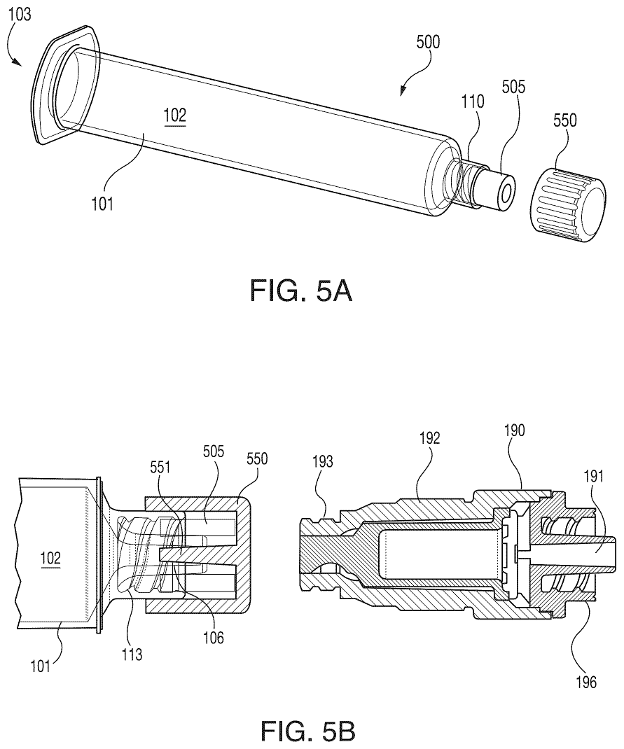

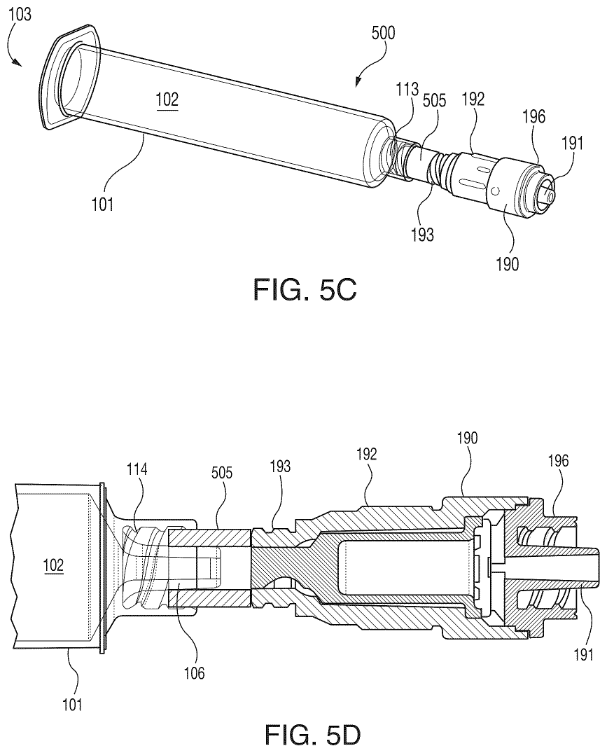

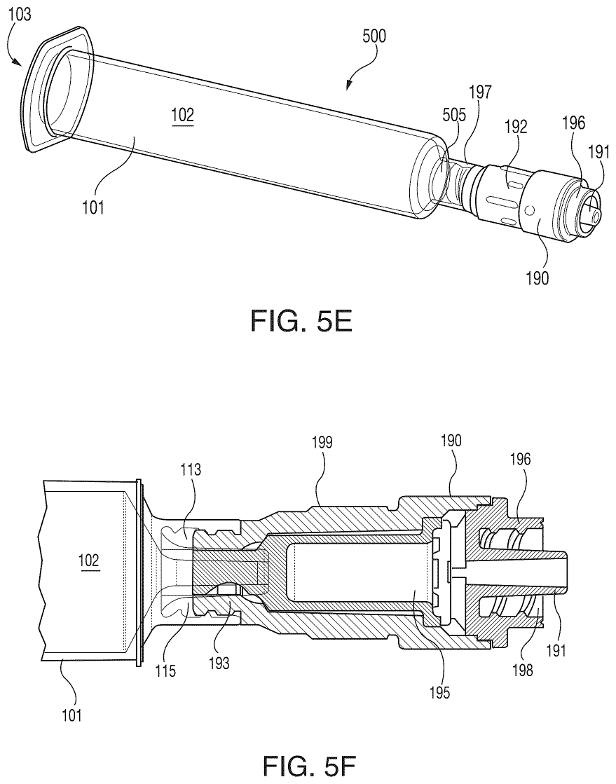

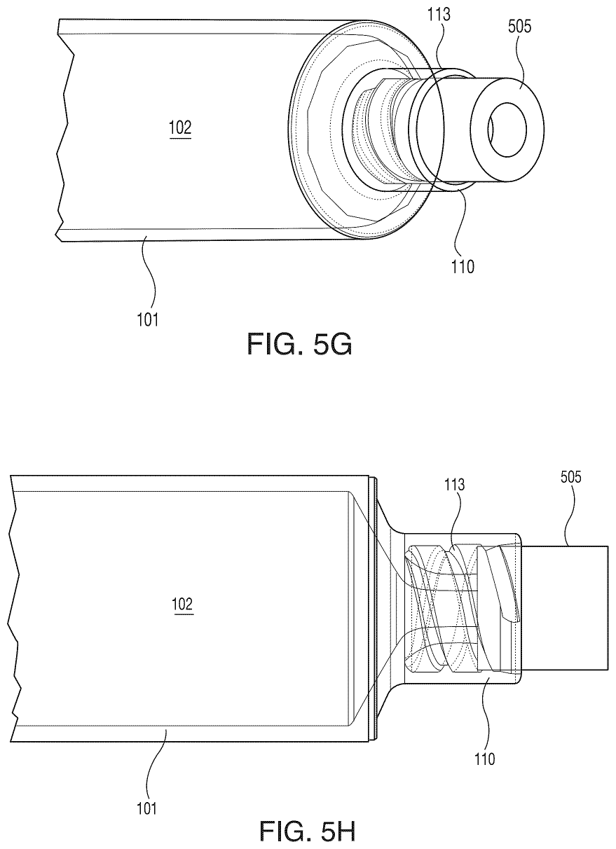

FIGS. 5A-H show another embodiment of an integrated flush syringe and swab assembly 500 including foam cylinder forming the integral swab 505 which is fitted into the integral collar 110 and may be threadably engaged with the internal thread 113. FIG. 5B shows the vascular access device connector 190 aligned with the distal tip 106 of the integrated flush syringe and swab assembly 500 with the removable cap 550 covering the distal tip 106. The removable cap 550 includes a central finger 55 configured to block disinfectant from entering the distal tip 106. A practitioner pushes the components together as shown in FIGS. 5C and 5D and rotates the vascular access device connector 190 with respect to the integrated flush syringe and swab assembly 500 to clean and disinfect the proximal end of the vascular access device connector. The user then connects the vascular access device connector 190 to the distal tip 106 and threadably engages the two components as shown in FIGS. 5E and 5F. FIGS. 5G and 5H show additional view of the integral swab 515 in the form of a foam cylinder integrated on the integral collar 110.

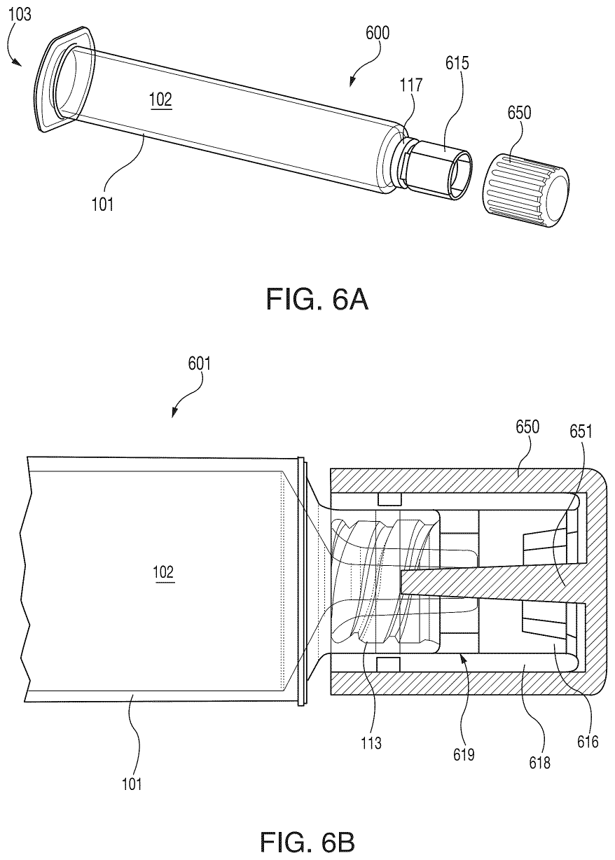

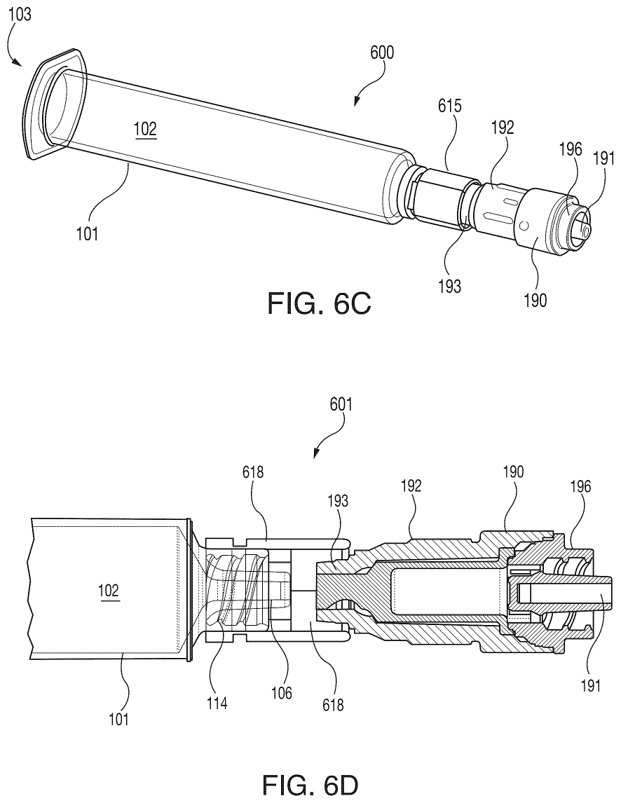

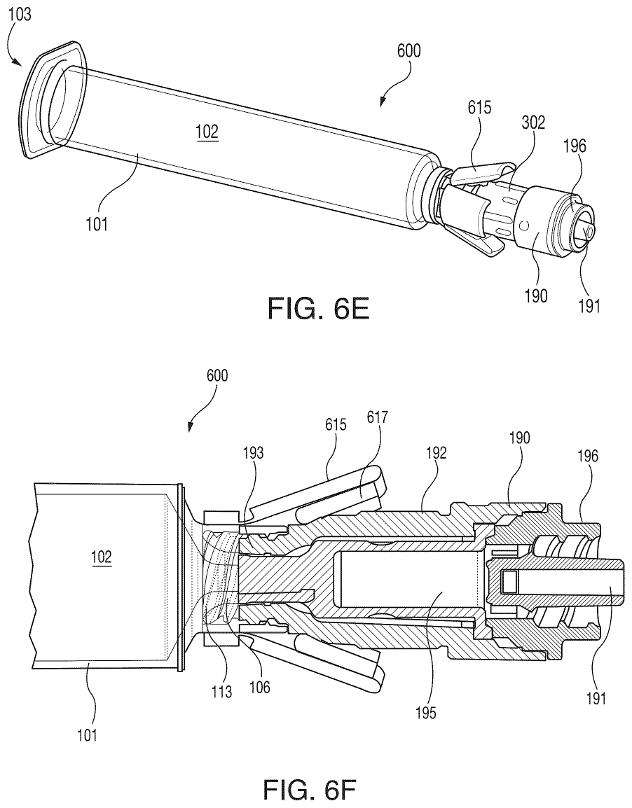

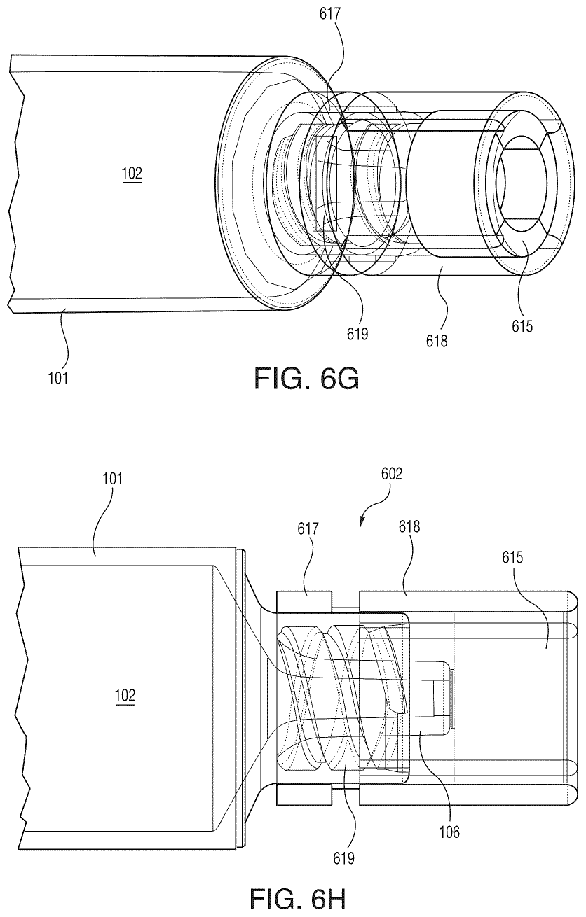

Referring now to FIGS. 6A-H, two embodiments of an integrated flush syringe and swab assembly 600 and 601 are shown. The integrated flush syringe and swab assembly 600 includes a segmented foam element that forms the swab 615. The segmented foam element that forms the swab 605 can include one or more slits to divide the swab into one, two, three, four, five, six or more separable projections as shown in FIG. 6A. This is similar to the slits and the separable projections described with respect to FIGS. 1A-F. A removable cap 650 includes a central finger 651 that prevents disinfectant or antimicrobial fluid from entering the distal tip 106.

In FIG. 6A, the swab 615 in the form of porous hollow mass comprising a hollow cylinder. In FIG. 6B, the integrated flush syringe and swab assembly 601 includes a clear plastic sleeve 618, which is provided to house the integral swab 616. The clear plastic sleeve 618 provides a window 619 configured to permit a user to view the syringe distal tip 106 when removing air bubbles from the flush syringe before a flushing procedure.

FIG. 6C shows the integrated flush syringe and swab assembly 6001 of FIG. 6A with a vascular access device connected thereto. FIG. 6D shows the integrated flush syringe and swab assembly 601 with the vascular access device connector 190 connected to the integrated flush and swab assembly 601.

FIGS. 6E and 6F show the integrated flush syringe and swab assembly 600 with the vascular access device connector 190 fully connected to the syringe, with the separable projections spread apart. In FIG. 6F, there is a foam insert 617 housed within the swab 615 to provide additional disinfection capability and to absorb additional disinfectant.

Referring now to FIGS. 6G and 6H and embodiment of an integrated flush syringe and swab assembly 602 is show with a swab 615 in the form of a foam cylinder mounted within a clear sleeve 618. Similar to the embodiment described with respect to FIG. 6B, a window 619 is provided, which permits a practitioner or other use of the device see bubbles in the distal tip before performing a flushing procedure. The foam cylinder has a height that is less than the height of the clear sleeve, and there is gap that provides the window 619 to see a portion of the distal tip 106 of the flush syringe.

An advantage provided by one or more embodiments is the provision of improved flush syringes to better ensure compliance with aseptic techniques, which ultimately reduces the chances of hospital acquired infections. Utilization of the one or more embodiments integrates the disinfectant and/or antimicrobial swab with the flush syringe to ensure compliance with cleaning procedures. The clinician will have to open fewer packages and does not have to carry alcohol swabs, and will be able to combine two steps into one, thus simplifying workflow. The clinician cannot skip or bypass the step of cleaning the connector because the integrated flush syringe and swab assemblies described herein forces the clinician to insert the vascular access device connector through the swab in the form of a porous mass that contains (e.g., soaked with or infused with) antimicrobial or disinfectant liquid.

While the present disclosure has been shown and described with reference to certain exemplary embodiments thereof, it will be understood by those skilled in the art that various changes in form and details may be made therein without departing from the spirit and scope of the embodiments of the present disclosure. Also, the inner and/or the outer housing of the disinfection cap can be single shot molded, or made by other suitable process. Furthermore, any of the features or elements of any exemplary implementations of the embodiments of the present disclosure as described above and illustrated in the drawing figures can be implemented individually or in any combination(s) as would be readily appreciated by skilled artisans without departing from the spirit and scope of the embodiments of the present disclosure.

In addition, the included drawing figures further describe non-limiting examples of implementations of certain exemplary embodiments of the present disclosure and aid in the description of technology associated therewith. Any specific or relative dimensions or measurements provided in the drawings other as noted above are exemplary and not intended to limit the scope or content of the inventive design or methodology as understood by artisans skilled in the relevant field of invention.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein has provided a description with reference to particular embodiments, it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure include modifications and variations that are within the scope of the appended claims and their equivalents.

What is claimed is:

1. A flush syringe and integrated swab assembly comprising:
a sidewall defining a barrel having an inside surface defining a chamber configured to retain a fluid, an open proximal end, a distal end including a distal wall with a tip extending distally therefrom having a passageway therethrough in fluid communication with the chamber;
a plunger rod comprising a proximal end and a distal end including a stopper slidably positioned in the chamber and configured to draw fluid into and drive fluid out of the chamber by movement of the stopper relative to the chamber, the plunger rod extending outwardly from the open proximal end of the chamber;
an integral collar extending from the distal wall of the barrel, surrounding the distal tip, and having a collar distal end and a collar proximal end, the integral collar including at least one sidewall having an inside surface including an inner thread thereon defining a Luer lock area and configured to connect to the hub of a vascular access device connector, the at least one sidewall further having an outside surface including an outer thread thereon configured for connection to a removable cap that when connected to the integral collar covers the integral collar to protect the distal tip and the collar from contamination; and
an integral swab comprising a porous hollow mass threadably connected of the integral collar, the porous hollow mass entrained with a disinfectant or an antimicrobial agent, wherein the removable cap is further configured to cover the integral swab.

2. The flush syringe and integrated swab assembly according to claim 1, wherein the integral swab is threadably connected to the inner thread of the integral collar.

3. The flush syringe and integrated swab assembly according to claim 1, wherein the porous hollow mass comprises a hollow cylinder having a proximal end and the porous hollow cylinder is threadably connected to the inner thread so that the proximal end of the hollow cylinder is spaced from the collar proximal end, providing a visible window between the proximal end of the hollow cylinder and the proximal end of the collar.

4. The flush syringe and integrated swab assembly according to claim 3, wherein the hollow cylinder comprises a height and a cross-sectional width, and there is at least one slit through the cross-sectional width defining at least two separable projections that are configured to separate upon insertion of a vascular access device connector and connection to the inner thread of the integral collar.

5. The flush syringe and integrated swab assembly according to claim 4, wherein the at least two separable projections are configured to contact the vascular access device connector on an outer peripheral surface and a distal surface of the vascular access device connector.

6. The flush syringe and integrated swab assembly according to claim 4, wherein there are at least two slits and the cross-sectional width defines at least four separable projections that are configured to separate upon insertion of a vascular access device connector and connection to the inner thread of the integral collar.

7. The flush syringe and integrated swab assembly according to claim 1, wherein the porous hollow mass comprises a crown portion and a skirt extending from crown portion, and there is at least one slit through the crown portion defining the at least two separable projections that are configured to separate upon insertion of a vascular access device connector and connection to the inner thread of the integral collar.

8. The flush syringe and integrated swab assembly according to claim 7, wherein the at least one slit extends through the skirt.

9. The flush syringe and integrated swab assembly according to claim 7, wherein there are two slits defining four separable projections that are configured to separate from each other upon insertion of a vascular access device connector and connection to the inner thread of the integral collar.

10. The flush syringe and integrated swab assembly according to claim 9, the two slits extend through the skirt.

11. The flush syringe and integrated swab assembly according to claim 7, wherein the crown portion comprises a plurality of bristles extending in a distal direction from the crown region.

12. The flush syringe and integrated swab assembly according to claim 7, wherein the crown and the plurality of bristles are configured to contact an end surface of a vascular access device connector.

13. The flush syringe and integrated swab assembly according to claim 12, wherein the removable cap comprises a central finger extending in a proximal direction that extends through the plurality of bristles and into the passageway of the distal tip to prevent entry of fluid into the chamber.

14. The flush syringe and integrated swab assembly according to claim 7, further comprising an auxiliary collar attached to the sidewall and the auxiliary collar including at least one hinge, paddles extending from the at least one hinge in a proximal direction, and the distal end of the collar is divided into a first section and a second section and wherein the paddles are configured to open the distal end of the collar by squeezing the paddles to expose the distal tip of the flush syringe.

15. The flush syringe and integrated swab assembly according to claim 14, wherein the auxiliary collar comprises distal end that is divided in a first section and a second section.

16. The flush syringe and integrated swab assembly according to claim 14, wherein the first section comprises a first hinge and the second section comprises a second hinge, and the first section and the second section are configured to pivot from a closed position to an open position to expose the distal tip of the flush syringe.

17. The flush syringe and integrated swab assembly according to claim 16, wherein the first section comprises a first lever and the second section comprises a second lever, the first lever and the second lever configured to respectively rotate the first section and the second section from a closed position to an open position to expose the distal tip of the flush syringe.

18. The flush syringe and integrated swab assembly according to claim 10, wherein the two slits through the skirt divide the skirt into four sections.

19. The flush syringe and integrated swab assembly according to claim 18, wherein upon connection of the vascular access device connector, the four sections of the skirt spread apart and contact a peripheral surface of the vascular access device connector.

20. The flush syringe and integrated swab assembly according to claim 19, wherein the skirt has a central opening.

21. The flush syringe and integrated swab assembly according to claim 18, wherein the four sections are housed in a clear plastic sleeve.
